(19) United States
(12) Patent Application Publication
(10) Pub. No.: US 2009/0241953 A1
(43) Pub. Date: Oct. 1, 2009

(54) VENTILATOR WITH PISTON-CYLINDER AND BUFFER VOLUME

(75) Inventors: Joseph Douglas Vandine, Newark, CA (US); Steve Vuong, Vista, CA (US); Iqbal Shahid, Mountain View, CA (US); Ravikumar V. Kudaravalli, Manassas, VA (US)

Correspondence Address: NELLCOR PURITAN BENNETT LLC;ATTN: IP LEGAL, 60 MIDDLETOWN AVENUE, NORTH HAVEN, CT, 06473, US

(73) Assignee: Nellcor Puritan Bennett LLC, Boulder, CO (US)

(21) Appl. No.: 12/414,430

(22) Filed: Mar. 30, 2009

Related U.S. Application Data
(60) Provisional application No. 61/041,083, filed on Mar. 31, 2008.

(51) Int. Cl. A61M 16/12

(52) U.S. Cl. 128/204.21; 128/205.18; 128/205.11

(57) ABSTRACT

A mechanical ventilator is provided with a piston-cylinder for performing an air displacement function and a buffer volume and associated output valve for providing an air metering function. The piston-cylinder may comprise a reciprocating arrangement, in which compressed air is supplied to the buffer volume with each stroke of the piston.

### RELATED APPLICATION

This application claims priority from U.S. Patent Application No. 61/041,083 which was filed on Mar. 31, 2008, and is incorporated herein by reference in its entirety.

### TECHNICAL FIELD

The present invention is generally directed to a mechanical ventilator. In particular, the present invention is directed to a mechanical ventilator with a reciprocating piston-cylinder that charges a buffer volume.

### BACKGROUND

Mechanical ventilators are used to provide a breathing gas to a patient who is unable to breathe without assistance. In modern medical facilities, pressurized air and oxygen sources are often available from wall outlets. Accordingly, mechanical ventilators may include pressure regulating valves connected to centralized sources of pressurized air and pressurized oxygen. The pressure regulating valves, which are typically proportional solenoids (PSOLs), function to regulate flow so that respiratory air having a desired concentration of oxygen is supplied to a patient at desired pressures and rates. However, centralized sources of pressurized air and oxygen are not always available. In addition, it is often desirable to provide a mechanical ventilator that is portable, or that can operate during an emergency when line power is not available or during periods when pressurized air and/or oxygen from a centralized source is otherwise not available.

With respect to a ventilator that is capable of operating independently of an external source of pressurized air, some mechanism for compressing air must be provided. For example, piston and bellows-based air delivery systems have been used in mechanical ventilators. As other examples, turbine based systems have been developed. However, all of these systems have disadvantages. For example, piston-based systems have been inefficient, because the frictional and pumping losses encountered during the separate intake and compression strokes require a significant amount of the work required to move the piston. In addition, the need for the piston to recover its position at the end of a stroke may disrupt gas delivery In systems that incorporate a bellows to provide a volume ventilator, the size of the apparatus is relatively large. Other systems, such as those that incorporate turbines, are limited in the amount of flow they can deliver against a load, and perform differently at different altitudes. Therefore, ventilators that use a turbine to pressurize respiratory air can be difficult to implement, particularly in connection with a portable device.

### SUMMARY

A mechanical ventilator is provided that, in one embodiment, incorporates a reciprocating piston-cylinder for performing an air displacement function and a buffer volume with a variable outlet valve for performing an air metering function. More particularly, a piston that is double acting in that it provides compressed air as an output in both directions of travel within a matching cylinder is provided. The air compressed by the piston is delivered to a buffer volume that is maintained at or about a selected pressure. The compressed air is released from the buffer volume in a controlled manner through the outlet valve for delivery to a patient.

In accordance with another embodiment of a mechanical ventilator device or method as described herein, the gas supplied to the patient is molecular oxygen-enriched. Accordingly, compressed air released from the buffer volume may be delivered to a mixing chamber. Molecular oxygen is admitted into the mixing chamber in an amount necessary to achieve the desired level of enrichment. Alternatively, oxygen may be admitted directly into the buffer volume rather than into a separate chamber. As yet another alternative embodiment, oxygen may be drawn into the piston-cylinder as part of one or both intake strokes of the piston-cylinder cycle. Accordingly, embodiments of the present invention may be used in association with an oxygen concentrator, as well as with a source of compressed oxygen.

In accordance with an embodiment of the present invention, a mechanical ventilator device is provided that includes: a motor; a cylinder, including a molecular oxygen-containing gas inlet and outlet; a piston, wherein the motor moves the piston within the cylinder to draw gas in and expel gas from the cylinder; a buffer volume in communication with the gas outlet of the cylinder, wherein the buffer volume holds pressurized gas delivered to the buffer volume from the gas outlet of the cylinder; and an outlet valve in communication with the buffer volume, wherein pressurized gas can be selectively released from the buffer volume by operation of the outlet valve.

In accordance with another embodiment of the present invention, a method for providing respiratory air to a patient is provided that includes compressing air by driving a piston within a cylinder; charging a buffer volume with compressed ambient air supplied from the reciprocation of the piston within the cylinder; and releasing compressed ambient air from the buffer volume for delivery to a patient.

In accordance with yet another embodiment of the present invention, a method for providing mechanical ventilation is provided that includes compressing air by driving a reciprocating piston, including in a first mode: moving the piston in a first direction within the cylinder; forcing air out of a first region of the cylinder on a first side of the piston through a first outlet port; drawing air into a second region of the cylinder on a second side of the piston through a first intake port; and in a second mode: moving the piston in a second direction within the cylinder; forcing air out of the second region of the cylinder on the second side of the piston through a second outlet port; drawing air into the first region of the cylinder on the first side of the piston through a second intake port; delivering the compressed air to a buffer volume; and releasing compressed air from the buffer volume through a variable valve.

Embodiments of the present invention can provide smaller pressure differentials across the piston, which can minimize gas leak past the piston, particularly when pressure within the buffer volume is relatively low (e.g., less than 10 psig). This in turn can lead to a lighter cylinder and buffer. Smaller pressure differentials across the piston and lower final pressures can permit the use of light duty piston seals and provide a long life due to lower wear rates and lower friction, and permit the use of a relatively low power motor and power supply.

Additional features and advantages of embodiments of the present invention will become more readily apparent from the following description, particularly when taken together with the accompanying drawings.

### BRIEF DESCRIPTION OF THE DRAWINGS

FIG. 1A is a depiction of a mechanical ventilator in accordance with embodiments of the present invention;

FIG. 1B is a depiction of a mechanical ventilator in accordance with other embodiments of the present invention;

FIG. 1C is a depiction of a mechanical ventilator in accordance with other embodiments of the present invention;

FIG. 2A is a depiction of a piston-cylinder valve and drive arrangement in accordance with embodiments of the present invention;

FIG. 2B is a depiction of a piston-cylinder valve and drive arrangement in accordance with other embodiments of the present invention;

FIG. 2C is a depiction of a piston-cylinder valve and drive arrangement in accordance with other embodiments of the present invention;

FIG. 3 is a depiction of control inputs and outputs associated with a controller of a mechanical ventilator in accordance with embodiments of the present invention; and

FIG. 4 is a flowchart depicting aspects of the operation of a mechanical ventilator in accordance with embodiments of the present invention.

### DETAILED DESCRIPTION

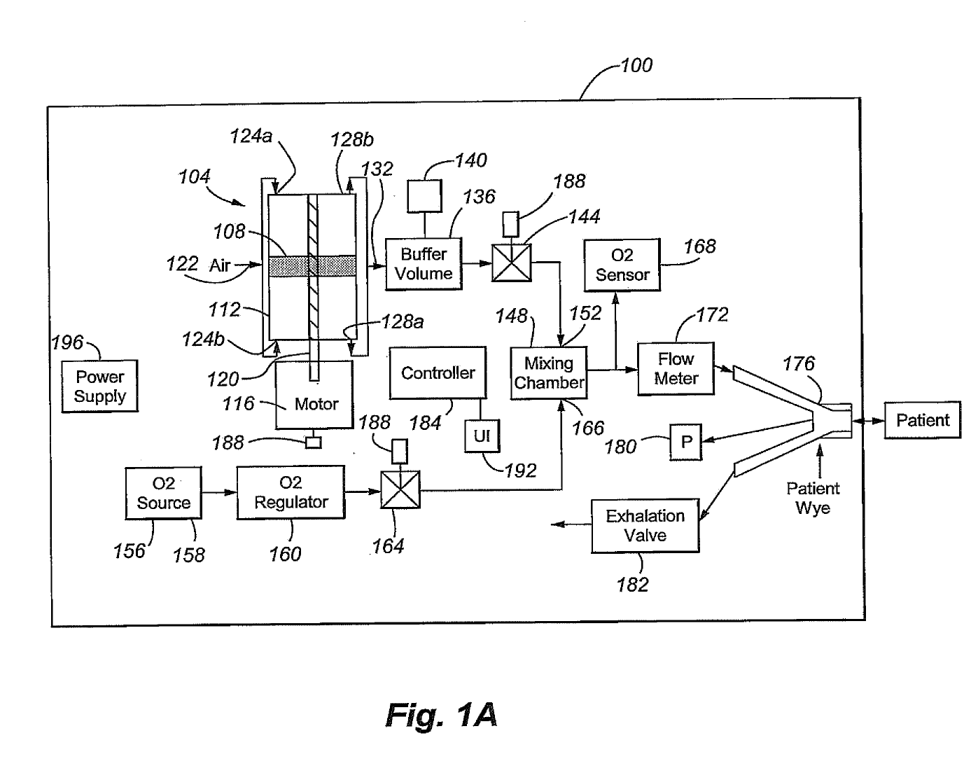

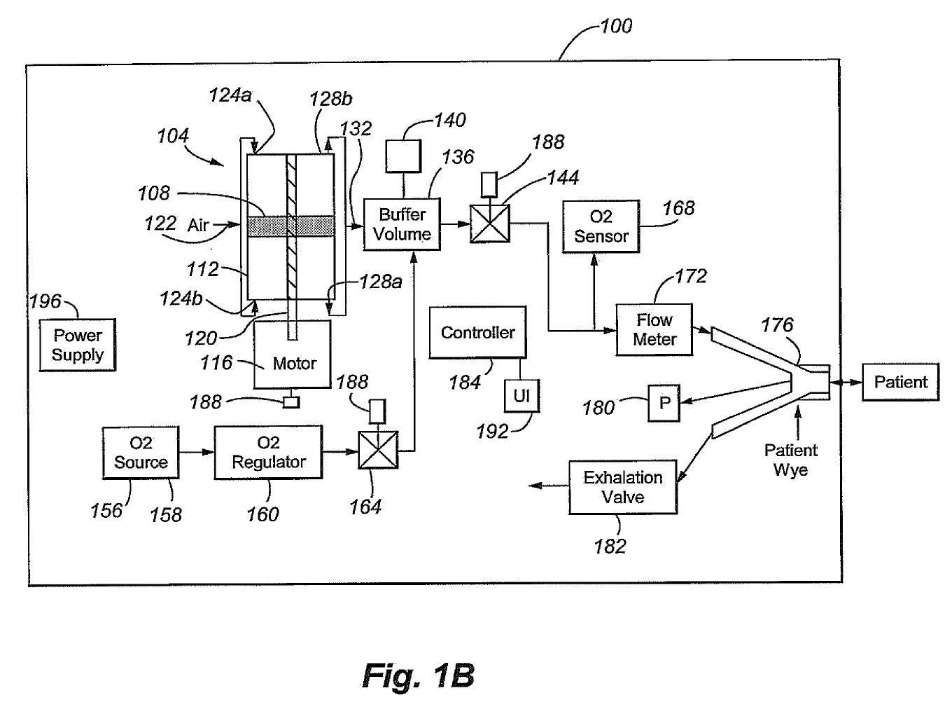

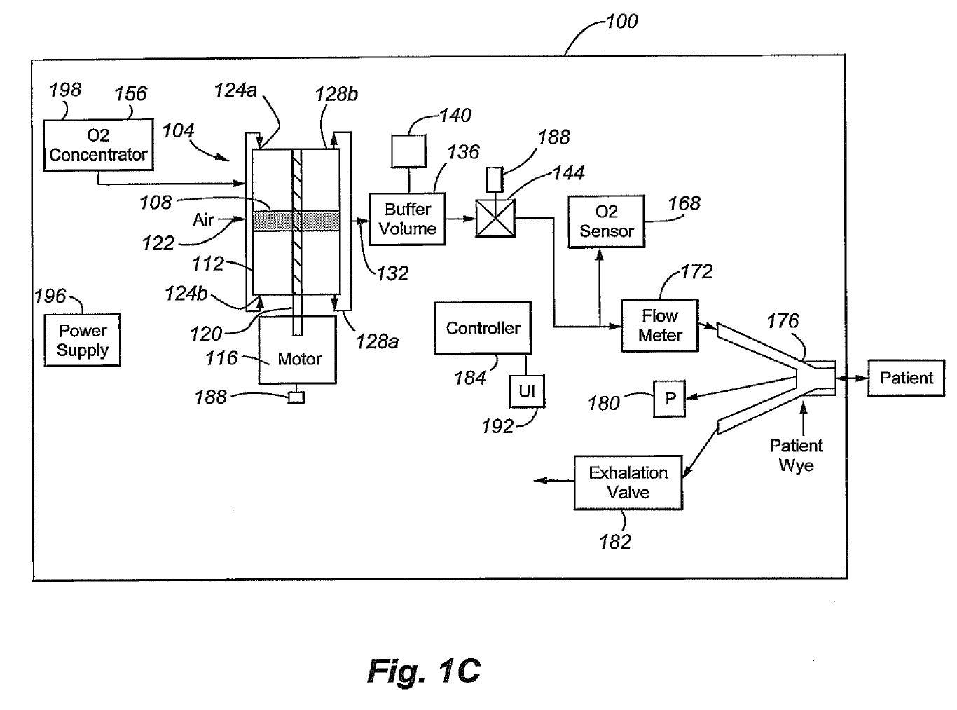

FIG. 1A depicts a piston-cylinder-based mechanical ventilator 100 with a buffer volume in accordance with embodiments of the present invention. In particular, the ventilator 100 includes a means for displacing a molecular oxygen-containing gas, that comprises a piston-cylinder 104 having a piston 108 that reciprocates within a cylinder 112. While the molecular oxygen-containing gas is described herein as being air, it is to be understood that the gas is not limited to air and may have a composition different from air. For example, the composition may be molecular oxygen-enriched relative to air, a helium-oxygen mixture (e.g., heliox), molecular-oxygen only, or other therapeutic gases. The piston 108 can be driven by a motor or means for driving 116 that turns a drive screw or worm gear 120 to move the piston 108 within the cylinder 112. While the piston 108 is being driven, air is drawn in through a gas inlet 122 and expelled through a gas outlet 132. More particularly, as the piston 108 is driven in a first direction, for example in a downward direction in FIG. 1A, air is drawn in a first intake port 124a and simultaneously expelled through a first outlet port 128a. As the piston 108 is driven in a second direction, for example in an upward direction in the figure, air is drawn into the cylinder 112 through a second intake port 124b and simultaneously expelled through a second outlet port 128b. Accordingly, the piston 108 performs useful work by forcing air through an outlet port 128 regardless of the direction that the piston is being driven in.

Air forced through one of the outlet ports 128 is delivered by the outlet 132 to a buffer volume or means for accumulating pressurized gas 136. A buffer volume pressure sensor 140 monitors the pressure within the buffer volume 136. As described in greater detail elsewhere herein, the motor 116 can be controlled so that the pressure within the buffer volume 136 is maintained at a desired level, which is commonly a level sufficient for a flow controller such as a proportional solenoid valve. A buffer volume outlet valve 144 is controlled to selectively release compressed air from the buffer volume 136. The buffer volume 136, buffer volume pressure sensor 140, and buffer volume outlet valve 144 generally comprise a means for metering air for delivery to a patient.

In accordance with the embodiment illustrated in FIG. 1A, the compressed air released from the buffer volume 136 by the buffer volume outlet valve 144 is delivered to a mixing chamber or means for mixing 148 through a first mixing chamber inlet 152. Oxygen supplied from a means for enriching the pressurized gas with molecular oxygen comprising an oxygen source 156, an oxygen regulator 160 and an oxygen flow valve 164 is supplied to the mixing chamber 148 through a second mixing chamber inlet 166. The oxygen source 156 may comprise a bottle, cylinder or other reservoir of compressed oxygen 158. The concentration of oxygen in the mixing chamber 148 is monitored by an oxygen sensor 168. The amount that the oxygen flow valve 164 is opened can be controlled so that a desired oxygen concentration is maintained in the mixing chamber 148. In some embodiments, the oxygen concentration in mixing chamber 148 may be controlled by volumetric measurement and/or metering of oxygen and air intake into chamber 148.

In accordance with the embodiment illustrated in FIG. 1B, molecular oxygen is supplied from an oxygen source 156, such as a cylinder of compressed oxygen 158, via an oxygen regulator 160 and an oxygen flow valve 164 to the buffer volume 136. Accordingly, in this alternate embodiment, the buffer volume 136 functions as a mixing chamber. An oxygen sensor 168 may sense the concentration of oxygen delivered from the buffer volume 136, and the oxygen flow valve 164 can be controlled to maintain the oxygen concentration at a desired level.

In accordance with the embodiment illustrated in FIG. 1C, molecular oxygen is drawn into the piston-cylinder 104 from an oxygen source 156 comprising an oxygen concentrator 198, together with ambient air. Accordingly, embodiments of the present invention can be used in association with an oxygen concentrator 198 or other unpressurized source of oxygen, and do not require use with a compressed source of oxygen.

A flow meter 172 can be provided to monitor flow rates as delivered to a patient wye 176. In addition, a pressure sensor 180 can be included to detect the pressure in the patient wye 176, so that remedial action can be taken and/or an alarm can be triggered should the pressure fall outside of normal parameters. The patient wye 176 also may incorporate an exhalation valve 182.

To provide a desired output to a patient, to detect and respond to conditions that are out of the ordinary, and to otherwise control the operation of the mechanical ventilator device 100, a central controller 184 can be provided. Alternatively or in addition, the mechanical ventilator device 100 can include a number of distributed or satellite controllers 188 to perform specific or limited functions. For example, each proportional solenoid or other valve 144, 164 and the motor 116 can be associated with a satellite controller 188. Control inputs may be entered by a clinician or the patient through a user input or interface 192. In addition, the mechanical ventilator 100 can incorporate a power supply 196. The power supply 196 can comprise a conduit for line power, a transformer, and/or a battery, fuel cell or other portable power source.

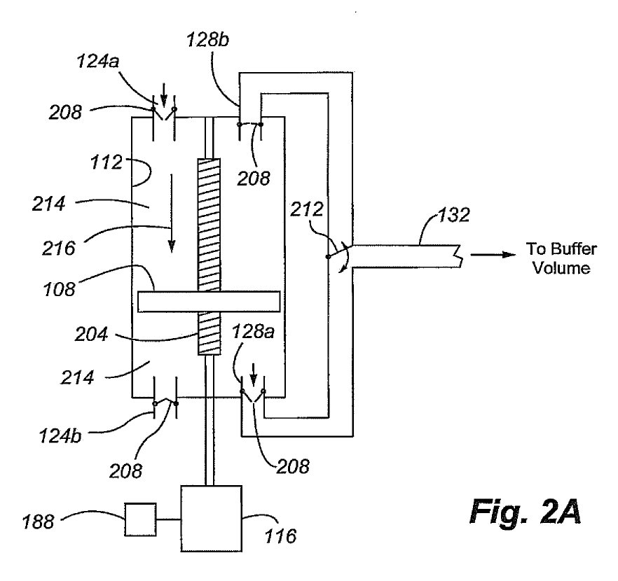

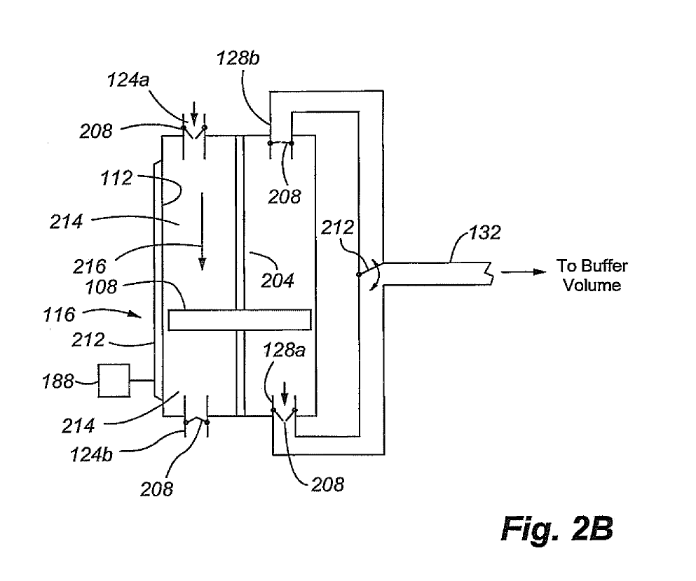

FIG. 2A depicts a piston-cylinder 104 valve and drive arrangement in accordance with embodiments of the present invention. In particular, a piston-cylinder 104 with a screw or worm gear 204 type drive is illustrated. The screw 204 is driven by the motor 116. By varying the speed at which the motor 116 turns the screw 204, the rate at which the piston 108 travels through the cylinder 112, and therefore the rate at which air is forced through the outlet 132, can be varied. In addition, the length of the piston 108 stroke within the cylinder 112 can be selected by selecting the location of the piston 108 within the cylinder 112 at which the rotation of the screw 204 is reversed. Therefore, the illustrated arrangement allows flow parameters at the outlet 132 to be varied.

As can be appreciated by one of skill in the art, other drive mechanisms can be employed. For example, as illustrated in FIG. 2B, an arrangement in which the cylinder 108 is driven magnetically can be provided. More particularly, the piston 108 can function as part of a linear induction motor 116 in which the piston 108 is driven by the magnetic field produced by windings 212. This arrangement also permits the speed and stroke length of the piston 108 within the cylinder 112 to be varied by varying the waveform of the magnetic field produced by the windings 212 to provide selected flow parameters at the outlet 132.

Yet another method for driving the piston 108, illustrated in FIG. 2C, involves coupling a radial drive arm or rod 220 from the center of the piston 108 to an eccentric point 224 on a circular drive wheel 228 and using a motor to rotate the wheel 228. The eccentric point or carriage roller 224 travels within a slot 232 found in a carriage 236. As can be appreciated by one of skill in the art, as the motor rotates the wheel 228 the eccentric point 224 travels back and forth within the slot 232, while causing the carriage 236 to travel in a reciprocating motion along and between a pair of carriage guides 240. The carriage 236 is connected to the piston rod 220 to cause the piston 108 to reciprocate within the cylinder 112 as the drive wheel 228 rotates. A rod bushing 244 provides a seal to prevent leakage of gas into or out of the cylinder 112 around the piston rod 220. In this embodiment, the flow can be controlled by varying the rotational speed of the wheel 228.

As shown in FIGS. 2A-2C, the total volume within the cylinder 112 is divided into two regions 214 by the cylinder 108. The total volume of the cylinder less the volume of the piston 108 and any associated drive mechanisms, such as the screw 204, along the length of the piston 108 stroke, generally defines the working volume of the piston-cylinder 104. As the piston 108 reciprocates within the cylinder 112, the volume of one region 214 increases while the volume of the other region 214 decreases, and while the working volume remains constant. This arrangement allows the piston-cylinder 104 to simultaneously draw air in through the intake 122 and expel air through the outlet 132. In addition, there are two inlet ports 124a-b and two outlet ports 128a-b in communication with the interior of the cylinder 112. More particularly, at least one inlet port 124 and at least one outlet port 128 are located at each end of the cylinder 112. This allows the piston 108 to draw air into an inlet port 124 and to force air out through an outlet port 128 in both directions of travel within the cylinder 112.

In particular, as shown in FIGS. 2A and 2B, in a first mode, while the piston 108 is traveling in a first direction (downward in the figures), air is drawn into the first intake port 124a and is simultaneously forced out of the first outlet port 128a. Meanwhile, the second intake port 124b and the second outlet port 128b are closed. The situation is reversed in a second mode. In the second mode, piston 108 is traveling in a second direction (opposite the first direction), air is drawn into the second intake port 124b and is simultaneously forced out the second outlet port 128b, while the first intake port 124a and the first outlet port 128a are closed. The reciprocating piston-cylinder 104 arrangement, by drawing with each stroke ambient air into the cylinder 112 on one side of the piston 108 and compressing and forcing air out the outlet 132 using the opposite side of the piston 108, provides increased efficiency as compared to designs that feature separate intake and compression strokes. In order to prevent unwanted back flows, each of the intake 124 and outlet 128 ports may incorporate check valves 208. A check valve 212 may also be incorporated at the tee where the outlet ports 128 are connected to the outlet 132. These check valves 208, 212 may be any of various designs, including simple reed valves, flap or butterfly valves, or actively operated valves.

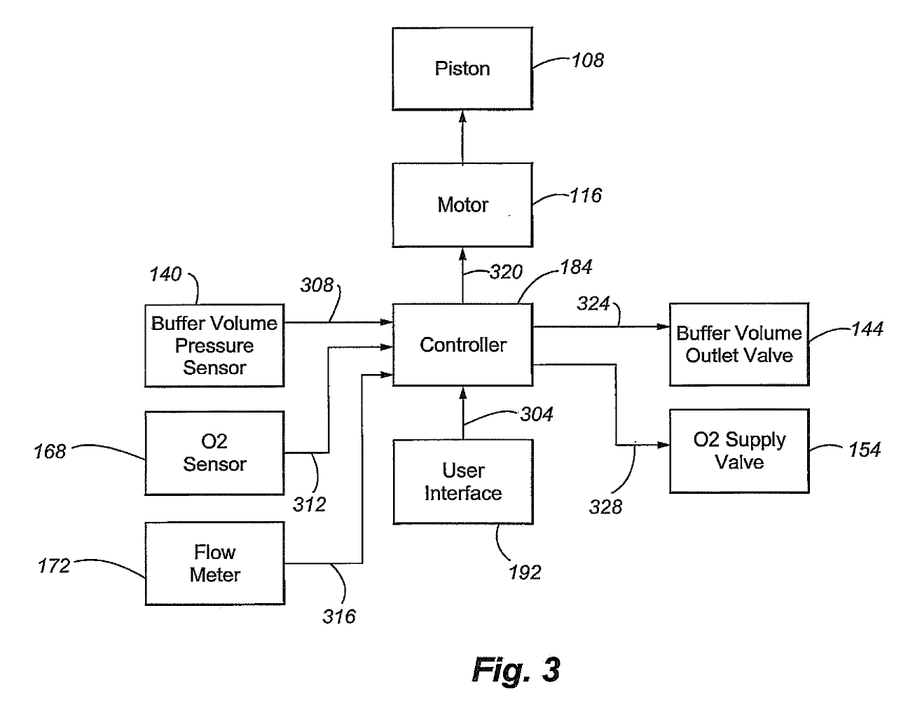

FIG. 3 depicts the relationships between various components of a mechanical ventilator in accordance with embodiments of the present invention. More particularly, control inputs and outputs to and from a controller 184 are illustrated. The inputs include a clinician input signal 304 provided through the user interface 192. The clinician input signal 304 generally specifies target respiratory parameters that are selected by a clinician or operator of the mechanical ventilator 104. The inputs to the controller 184 also include a pressure signal 308 provided by the buffer volume pressure sensor 140, an oxygen concentration signal 312 provided by the oxygen sensor 168, and a flow rate signal 316 provided by the flow meter 172. Outputs from the controller 184 include a motor control signal 320, a buffer volume outlet valve control signal 324, and an oxygen supply valve control signal 328. For simplicity of illustration, the various input and output signals are shown in association with a central controller 184. However, as can be appreciated by one of skill in the art, some or all of the signals can be associated with satellite controllers 188.

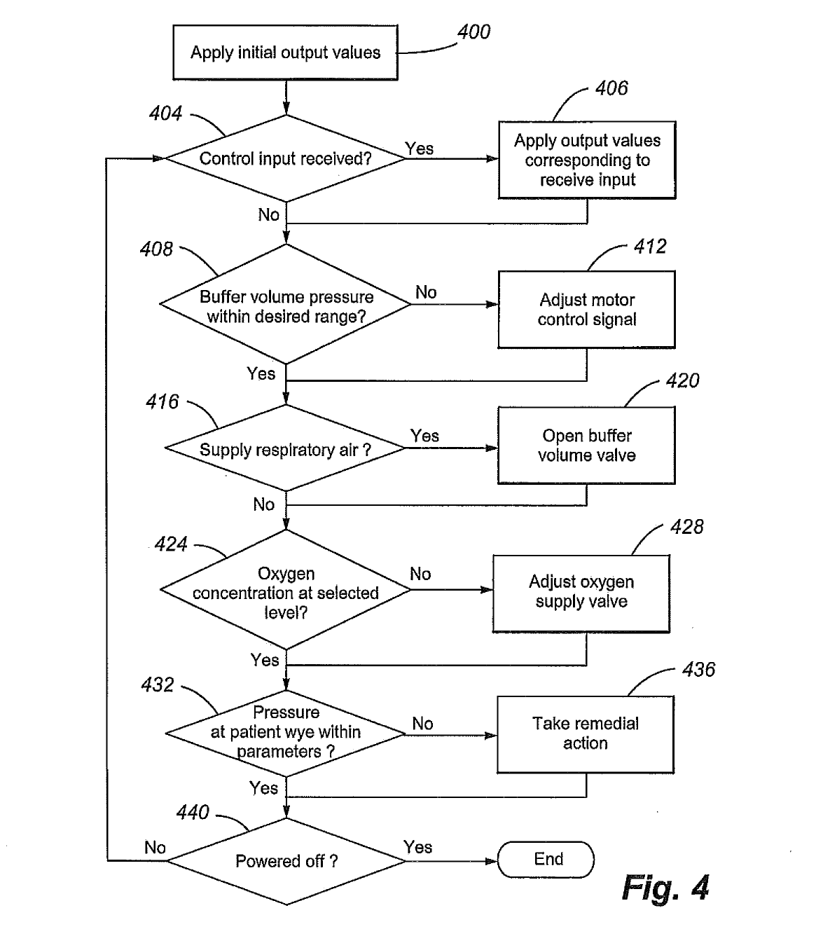

FIG. 4 is a flowchart depicting aspects of the operation of a mechanical ventilator 100 in accordance with embodiments of the present invention. Upon start up, initial output parameter settings and corresponding initial control output values for the motor 116 and valves 144, 164 may be applied (step 400). At step 404, a determination is made as to whether clinician or control input 104 has been received. If clinician input is received, output values corresponding to that input are applied (step 406).

A determination may next be made as to whether the pressure of the air inside the buffer volume 136 is within the desired range (step 408). In general, the buffer volume pressure is maintained within a relatively small range of pressures. If the buffer volume pressure is outside of the desired range, the motor control signal 328 can be varied accordingly (step 412). For example, if the pressure in the buffer volume 136 is below the desired pressure, the speed at which the piston 108 moves within the cylinder 112 can be increased by increasing the speed at which the motor 116 rotates the drive screw. In a typical arrangement, the rate of reciprocation of the piston 108 within the cylinder 112 will be much greater than the rate of the patient's respiratory cycle. In addition, as can be appreciated by one of skill in the art, the pressure within the buffer volume will vary with the respiratory cycle of the patient. The length of the piston 108 stroke within the cylinder 112 can also be varied. Also, the speed at which the piston 108 moves within the cylinder 112 can be controlled so that it is different at different points in the piston stroke. Therefore, the output of the piston-cylinder 104 can be tailored to the respiratory cycle of the patient so that a consistent or desired pressure within the buffer volume 136 is maintained. As an example, and without necessarily importing limitations into the claims, the air within buffer volume 136 can be maintained at a pressure of less than 15 psig. As a further non-limiting example, the pressure of the air within the buffer volume 136 can be maintained at about 7 psig. As still another non-limiting example, the pressure of the air within the buffer volume 136 can be maintained at about 3 psig. In some embodiments, the valve controller can compensate for changes in buffer pressure of at least several psi.

The buffer volume 136 generally functions as a reservoir of compressed air that, enriched with oxygen, will be supplied to the patient. As can be appreciated by one of skill in the art, in a mechanical ventilator, pressurized air is supplied to the patient during a period of time corresponding to the inspiratory portion of normal breathing. In accordance with embodiments of the present invention, the flow of respiratory air from the buffer volume 136 is controlled by the buffer volume valve 144. In particular, in response to determining that respiratory air should be supplied to the patient (step 416), the buffer volume valve 144 is opened (step 420). The rate of flow of respiratory air to the patient can be controlled and shaped as desired by controlling the opening of the buffer volume valve 144. Moreover, because the supply of compressed air to the buffer volume 136 by the piston-cylinder 104 can be varied by the controller 184, precise control of the respiratory air supplied to the patient can be achieved. Feedback regarding the actual flow of respiratory air being supplied to the patient is provided by the flow meter 172 and can be used by the controller 184 to adjust the opening of the buffer volume valve 144.

Another parameter that can be controlled during operation of the mechanical ventilator 104 is the concentration of molecular oxygen in the air delivered to the patient through the patient wye 176. The concentration of molecular oxygen is generally selected to be some percentage of the respiratory air delivered to the patient, which is sensed by the oxygen sensor 168. If the desired oxygen concentration is not present in the respiratory air (step 424), as measured by the oxygen sensor 168, the controller 184 can change the opening of the oxygen supply valve 164 the oxygen supply signal 328 (step 428).

At step 432, a determination may be made as to whether the pressure in the patient wye 176, as sensed by the pressure sensor 180, is within specified parameters. If the pressure falls outside of the desired parameters, remedial action can be taken (step 436), such as sounding an alarm or adjusting the buffer volume outlet valve 144.

A determination may next be made as to whether the mechanical ventilator 100 has been powered off (step 440). If the mechanical ventilator 100 has been powered off the process may end. If the mechanical ventilator has not been powered off, the process may return to step 404. Although FIG. 4 depicts aspects of the operation of a mechanical ventilator in accordance with embodiments of the present invention as a set of different operations that are performed in series, it should be appreciated that embodiments of the present invention are not so limited. For example, in a typical implementation, the receipt of signals at a controller 184, 188, and/or the generation of output signals by a controller 184, 188, can occur in any sequence or even simultaneously.

From the description provided herein, it can be appreciated that embodiments of the present invention provide a mechanical ventilator 100 in which the air displacement function is performed by a reciprocating piston-cylinder 104. Moreover, the piston-cylinder 104 can be operated under a relatively light load, because the pressure at which the buffer volume 136 is charged is relatively low (e.g., less than 15 psig). The use of a reciprocating piston-cylinder 104, which provides both compressed air and draws in air for subsequent compression with each stroke, and operation of the piston-cylinder 104 at relatively light pressures, can provide improved efficiency as compared to arrangements in which intake and compression strokes are performed separately and that are operated at higher pressures. In addition, embodiments of the present invention provide a buffer volume 136 for accumulating pressurized air supplied by the piston-cylinder 104. Moreover, the buffer volume 136 can be charged with air provided by a source other than a piston-cylinder that provides compressed air with each stroke, such as a conventional piston-cylinder or a turbine. According to embodiments of the present invention, air is metered out of the buffer volume 136 for delivery to the patient. The metering function can be performed by a controller 184 operated valve 144. In accordance with embodiments of the present invention, the buffer volume outlet valve 144 may comprise a proportional solenoid (PSOL), a motor controlled valve, or some other type of variable orifice device. Other valves included in the mechanical ventilator 100, such as the oxygen supply valve 164) may also comprise a PSOL type valve, a motor controlled valve, or some other type of variable orifice device.

The foregoing discussion of the invention has been presented for purposes of illustration and description. Further, the description is not intended to limit the invention to the form disclosed herein. Consequently, variations and modifications commensurate with the above teachings, within the skill or knowledge of the relevant art, are within the scope of the present invention. The embodiments described hereinabove are further intended to explain the best mode presently known of practicing the invention and to enable others skilled in the art to utilize the invention in such or in other embodiments and with the various modifications required by their particular application or use of the invention. It is intended that the appended claims be construed to include alternative embodiments to the extent permitted by the prior art.

What is claimed is:

1. A mechanical ventilator device, comprising:
a motor;
a cylinder, including;
a gas inlet;
a gas outlet;
a piston, wherein the motor moves the piston within the cylinder to draw gas in and expel gas from the cylinder;
a buffer volume in communication with the gas outlet of the cylinder, wherein the buffer volume holds pressurized gas delivered to the buffer volume from the gas outlet of the cylinder; and
an outlet valve in communication with the buffer volume, wherein pressurized gas can be selectively released from the buffer volume by operation of the outlet valve.

2. The device of claim 1, further comprising:
first and second intake ports, wherein the gas inlet is in communication with the first and second intake ports,
first and second outlet ports, wherein the gas outlet is in communication with the first and second outlet ports,
wherein the first intake port and the second outlet port are in communication with a first region within the cylinder on a first side of the piston;
wherein the second intake and first outlet port are in communication with a second region within the cylinder on a second side of the piston;
wherein in a first mode the piston is moved in a first direction, the first intake port and the first outlet port are open, and the second intake port and the second outlet port are closed; and
wherein in a second mode the piston is moved in a second direction, the first intake port and the first outlet port are closed, and the second intake port and the second outlet port are open.

3. The device of claim 1, further comprising:
a buffer volume pressure sensor operable to determine a pressure of gas contained within the buffer volume.

4. The device of claim 3, further comprising:
a first controller, wherein at least one of a speed and a frequency of the piston is modulated to deliver a desired flow of gas to the buffer volume and to maintain a desired pressure within the buffer volume.

5. The device of claim 4, further comprising:
a buffer volume outlet valve, wherein a desired flow of gas from the buffer volume is selectively provided, wherein the buffer volume outlet valve is variable, and wherein operation of the buffer volume outlet valve is controlled by at least one of the first controller or a second controller.

6. The device of claim 5, further comprising:
an oxygen source, wherein the oxygen source is pressurized; and
an oxygen source supply valve, wherein the oxygen source control valve is controlled by at least one of the first or second controllers or a third controller.

7. The device of claim 6, further comprising:
a mixing chamber, wherein the mixing chamber receives pressurized gas from the buffer volume and oxygen from the oxygen source;
an oxygen sensor in communication with an interior of the mixing chamber, wherein an output from the oxygen sensor is provided to at least one controller;
a flow meter at an outlet of the mixing chamber, wherein a signal output from the flow meter is provided to at least one controller; and
a patient wye in communication with the outlet of the mixing chamber.

8. The device of claim 5, further comprising:
an oxygen source in communication with the inlet to the cylinder, wherein molecular oxygen from the oxygen source is drawn into the cylinder by operation of the piston.

9. The device of claim 1, wherein the pressure across the piston is less than 15 psig.

10. A method for providing respiratory air to a patient, comprising:
compressing a molecular oxygen-containing gas by driving a reciprocating piston within a cylinder;
charging a buffer volume with compressed molecular oxygen-containing gas supplied from the reciprocation of the piston within the cylinder; and
releasing compressed molecular oxygen-containing gas from the buffer volume for delivery to a patient.

11. The method of claim 10, wherein molecular oxygen-containing gas is compressed and the buffer volume is charged with compressed molecular oxygen-containing gas when the piston is moved in a first direction within the cylinder, wherein molecular oxygen-containing gas is compressed and the buffer volume is charged with compressed molecular oxygen-containing gas when the piston is moved in a second direction within the cylinder, and wherein the first direction is opposite the second direction.

12. The method of claim 11, further comprising:
drawing molecular oxygen from an oxygen source and mixing the molecular oxygen and molecular oxygen-containing gas within the cylinder prior to delivering the compressed ambient molecular oxygen-containing gas and oxygen to the buffer volume.

13. The method of claim 11, further comprising:
injecting molecular oxygen from an oxygen source into the buffer volume, wherein the compressed molecular oxygen-containing gas is enriched with molecular oxygen prior to delivery to the patient.

14. The method of claim 11, further comprising:
delivering the compressed molecular oxygen-containing gas released from the buffer volume to a mixing chamber; and
injecting molecular oxygen from an oxygen source into the mixing chamber, wherein the compressed molecular oxygen-containing gas is enriched with oxygen prior to delivery to the patient.

15. The method of claim 10, wherein the molecular oxygen-containing gas compressed by driving a piston within a cylinder is ambient air.

16. The method of claim 10, wherein the buffer volume is charged to a pressure of less than 8 psig.

17. A method for providing mechanical ventilation, comprising:
compressing molecular oxygen-containing gas by driving a reciprocating piston, including:
in a first mode:
moving the piston in a first direction within the cylinder;
forcing compressed molecular oxygen-containing gas out of a second region of the cylinder on a second side of the piston through a first outlet port;
drawing molecular oxygen-containing gas into a first region of the cylinder on a first side of the piston through a first intake port;
in a second mode:
moving the piston in a second direction within the cylinder;
forcing compressed molecular oxygen-containing gas out of the first region of the cylinder on the first side of the piston through a second outlet port;
drawing molecular oxygen-containing gas into the second region of the cylinder on the second side of the piston through a second intake port;
in both the first and second modes, delivering the compressed molecular oxygen-containing gas to a buffer volume; and
releasing compressed molecular oxygen-containing gas from the buffer volume through a variable valve.

18. The method of claim 17, further comprising:
enriching the compressed molecular oxygen-containing gas with molecular oxygen by mixing the compressed molecular oxygen-containing gas with molecular oxygen from a compressed source.

19. The method of claim 18, wherein the molecular oxygen and the compressed molecular oxygen-containing gas are mixed in a mixing chamber that is separate from the buffer volume.

20. The method of claim 17, further comprising:
enriching the compressed molecular oxygen-containing gas with molecular oxygen by drawing oxygen into the cylinder together with ambient molecular oxygen-containing gas.
